(19) United States
(12) Patent Application Publication
(10) Pub. No.: US 2007/0276247 A1
(43) Pub. Date: Nov. 29, 2007

(54) Systems and methods for ultrasound imaging using an inertial reference unit

(76) Inventors: Vikram Chalana, Mill Creek, WA (US); Gerald McMorrow, Kirkland, WA (US); Jongtae Yuk, Redmond, WA (US)

Correspondence Address: BLACK LOWE & GRAHAM, PLLC, 701 FIFTH AVENUE, SUITE 4800, SEATTLE, WA, 98104, US

(21) Appl. No.: 11/222,360

(22) Filed: Sep. 8, 2005

Related U.S. Application Data
(63) Continuation-in-part of application No. 11/119,355, filed on Apr. 29, 2005.
(63) Continuation-in-part of application No. 10/701,955, filed on Nov. 5, 2003, now Pat. No. 7,087,022.
Application No. 10/701,955 is a continuation-in-part of application No. 10/443,126, filed on May 20, 2003, now Pat. No. 7,041,059.
(63) Continuation-in-part of application No. 11/061,867, filed on Feb. 17, 2005.
(63) Continuation-in-part of application No. 10/704,966, filed on Nov. 12, 2003, now Pat. No. 6,803,308.
(63) Continuation-in-part of application No. PCT/US03/24368, filed on Aug. 1, 2003.
(63) Continuation-in-part of application No. PCT/US03/14785, filed on May 9, 2003.
Application No. PCT/US03/14785 is a continuation of application No. 10/165,556, filed on Jun. 7, 2002, now Pat. No. 6,676,605.
(63) Continuation-in-part of application No. 10/633,186, filed on Jul. 31, 2003, now Pat. No. 7,004,904.
(60) Provisional application No. 60/609,184, filed on Sep. 10, 2004.
(60) Provisional application No. 60/608,426, filed on Sep. 9, 2004.
(60) Provisional application No. 60/621,349, filed on Oct. 22, 2004.
(60) Provisional application No. 60/423,881, filed on Nov. 5, 2002.
(60) Provisional application No. 60/400,624, filed on Aug. 2, 2002.
(60) Provisional application No. 60/423,881, filed on Nov. 5, 2002.

(30) Foreign Application Priority Data
Dec. 24, 2002 (KR) 10-2002-0083525

(51) Int. Cl. A61B 8/14

(52) U.S. Cl. 600/447; 128/916; 600/407

(57) ABSTRACT

Systems and methods for ultrasound imaging using an inertial reference unit are disclosed. In one embodiment, an ultrasound imaging system includes an ultrasound unit configured to ultrasonically scan at least one plane within a region of interest in a subject and generate imaging information from the scan. An inertial reference unit is provided that detects relative positions of the ultrasound unit as the ultrasound unit scans a plurality of plane. A processing unit is configured to receive the imaging information and the corresponding detected position and is operable to generate images of the region of interest.

### PRIORITY CLAIM

This application claims priority to U.S. provisional patent application Ser. No. 60/609,184 filed Sep. 10, 2004. This application also claims priority to U.S. provisional patent application Ser. No. 60/608,426 filed Sep. 9, 2004.

This application is a continuation-in-part of and claims priority to U.S. patent application filed Aug. 26, 2005 under U.S. Express Mail No. EV509173452US.

This application claims priority to U.S. provisional patent application Ser. No. 60/571,797 filed May 17, 2004. This application claims priority to U.S. provisional patent application Ser. No. 60/571,799 filed May 17, 2004.

This application claims priority to and is a continuation-in-part of U.S. patent application Ser. No. 11/119,355 filed Apr. 29, 2005, which claims priority to U.S. provisional patent application Ser. No. 60/566,127 filed Apr. 30, 2004. This application also claims priority to and is a continuation-in-part of U.S. Patent application Ser. No. 10/701,955 filed Nov. 5, 2003, which in turn claims priority to and is a continuation-in-part of U.S. patent application Ser. No. 10/443,126 filed May 20, 2003.

This application claims priority to and is a continuation-in-part of U.S. patent application Ser. No. 11/061,867 filed Feb. 17, 2005, which claims priority to U.S. provisional patent application Ser. No. 60/545,576 filed Feb. 17, 2004 and U.S. provisional patent application Ser. No. 60/566,818 filed Apr. 30, 2004.

This application is also a continuation-in-part of and claims priority to U.S. patent application Ser. No. 10/704,966 filed Nov. 10, 2004.

This application is a continuation of and claims priority to U.S. provisional patent application Ser. No. 60/621,349 filed Oct. 22, 2004.

This application is a continuation-in-part of and claims priority to PCT application serial number PCT/US03/24368 filed Aug. 1, 2003, which claims priority to U.S. provisional patent application Ser. No. 60/423,881 filed Nov. 5, 2002 and U.S. provisional patent application Ser. No. 60/400,624 filed Aug. 2, 2002.

This application is also a continuation-in-part of and claims priority to PCT Application Serial No. PCT/US03/14785 filed May 9, 2003, which is a continuation of U.S. patent application Ser. No. 10/165,556 filed Jun. 7, 2002.

This application is also a continuation-in-part of and claims priority to U.S. patent application Ser. No. 10/888,735 filed Jul. 9, 2004.

This application is also a continuation-in-part of and claims priority to U.S. patent application Ser. No. 10/633,186 filed Jul. 31, 2003 which claims priority to U.S. provisional patent application Ser. No. 60/423,881 filed Nov. 5, 2002 and to U.S. patent application Ser. No. 10/443,126 filed May 20, 2003 which claims priority to U.S. provisional patent application Ser. No. 60/423,881 filed Nov. 5, 2002 and to U.S. provisional application 60/400,624 filed Aug. 2, 2002. This application also claims priority to U.S. provisional patent application Ser. No. 60/470,525 filed May 12, 2003, and to U.S. patent application Ser. No. 10/165,556 filed Jun. 7, 2002. All of the above applications are herein incorporated by reference in their entirety as if fully set forth herein.

### FIELD OF THE INVENTION

This invention relates generally to ultrasound imaging, and more specifically, to systems and methods for ultrasound imaging using inertial reference units.

### SUMMARY OF THE INVENTION

The disclosed embodiments of the present invention are directed to systems and methods for ultrasound imaging using an inertial reference unit. In one aspect, an ultrasound imaging system includes an ultrasound unit configured to ultrasonically scan a plurality of planes within a region of interest in a subject and generate imaging information from the scans. An inertial reference unit is provided that detects relative positions of the ultrasound unit as the ultrasound unit scans the plurality of planes. A processing unit is configured to receive the imaging information and the corresponding detected positions and is operable to generate three dimensional images of the region of interest.

### BRIEF DESCRIPTION OF THE DRAWINGS

FIG. 1 is a block diagrammatic view of an ultrasound;

FIG. 1A is a side elevation view of an ultrasound transceiver that includes an inertial reference unit;

FIG. 1B is a side elevation view of an ultrasound transceiver that includes an inertial reference unit;

FIG. 1C is a side elevation view of an ultrasound transceiver that includes an inertial reference unit;

FIG. 1D is a side elevation view of an ultrasound transceiver that includes an inertial reference-unit contained within a detachable collar;

FIG. 1E is side elevation view of another ultrasound transceiver that includes an inertial reference unit contained within a detachable collar;

FIG. 2A is a schematic illustration of the accelerometer of the transceivers 10A-10E of FIGS. 1A-1E, respectively;

FIG. 2B is an expansion of the schematic illustration of FIG. 2A;

FIG. 3A is a schematic illustration of a gyroscope of transceivers 10A-10E of FIGS. 1A-1E, respectively;

FIG. 3B is an expansion of the schematic illustration of FIG. 3A;

FIG. 4 is a graphical representation of three dimensional (3D) distributed scan lines emanating from a transceiver that cooperatively form a scan cone;

FIG. 5A is a graphical representation of a plurality of scan planes that form a three-dimensional (3D) array having a substantially conical shape;

FIG. 5B is a graphical representation of scan plane;

FIG. 5C a graphical representation of a plurality of scan lines emanating from a hand-held ultrasound transceiver forming a single scan plane cross-sectioning through portions of an organ;

FIG. 5D is an isometric view of an ultrasound scan cone that projects outwardly from the transceivers of FIGS. 1A-E;

FIG. 5E is a top plan view of the scan cone 40 of FIG. 5D;

FIG. 6 is a schematic depiction of a transceiver housed in a cradle equipped for wireless communication;

FIG. 7 is a schematic depiction of a transceiver housed in a cradle equipped for cabled communication;

FIG. 8 is an isometric view of an inertial ultrasound imaging system using the transceiver of FIG. 1B applied to a side abdominal region of a patient;

FIG. 9 is an isometric view of an inertial ultrasound imaging system using the transceiver of FIG. 1B applied to a center abdominal region of a patient;

FIG. 10 is an isometric view of an inertial ultrasound imaging system using the transceiver of FIG. 1C applied to a center abdominal region of a patient;

FIG. 11 is an isometric view of an inertial ultrasound imaging system using the transceiver of FIG. 1A housed in a cradle configured for wireless communication;

FIG. 12 is an isometric view of an inertial ultrasound imaging system using the transceiver of FIG. 1A housed in a cradle configured for electrical cable communication;

FIG. 13 is a schematic illustration of a server-accessed local area network in communication with the inertial ultrasound imaging systems of FIGS. 9-12;

FIG. 14 is a schematic illustration of the Internet in communication with the inertial ultrasound imaging systems of FIGS. 9-12;

FIG. 15A is a schematic illustration of inertial reference coordinates superimposed over a transceiver experiencing translation changes between two transceiver locations regions;

FIG. 15B is an illustration that will be used to further describe the operation of the transceiver 10A of FIGS. 1A and 15A as a series of translation movements from an initial freehand position;

FIG. 16A is a schematic illustration of inertial reference coordinates superimposed over a transceiver experiencing rotation and tilt changes between two transceiver locations regions;

FIG. 16B is a schematic illustration that will be used to further describe the method of FIG. 16A involving a series of translation and rotation movements from an initial freehand position;

FIG. 17 is a flowchart that will be used to describe a method of forming a three dimensional ultrasound image, according to an embodiment of the invention. a method algorithm of the particular embodiments; and

FIG. 18 is a flowchart that will be used to further describe the method of FIG. 17, an expansion of sub algorithm 212 from FIG. 16.

### DETAILED DESCRIPTION OF THE PREFERRED EMBODIMENT

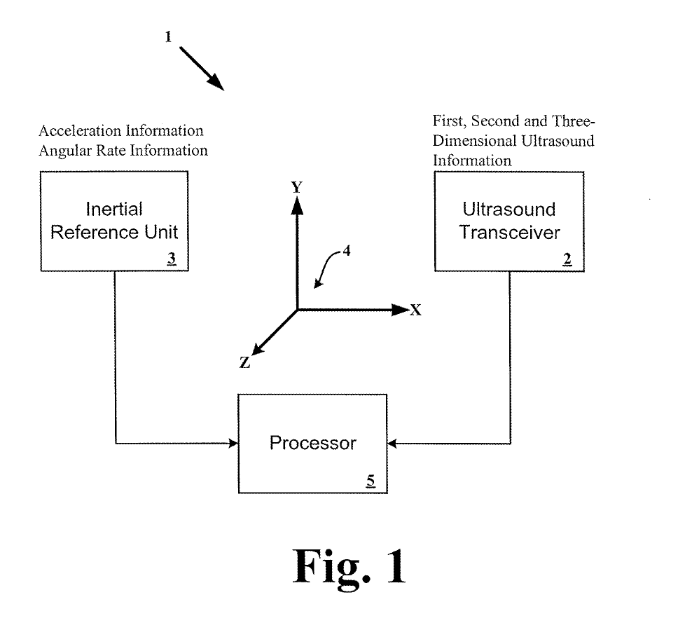

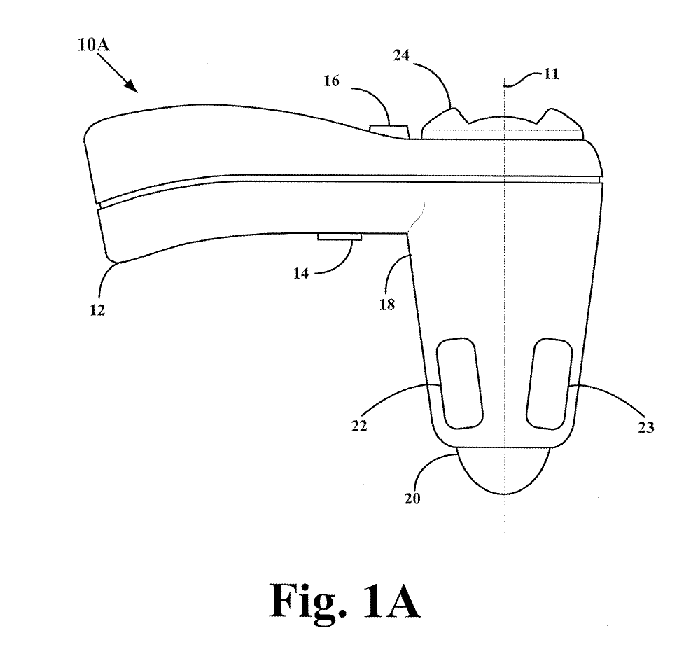

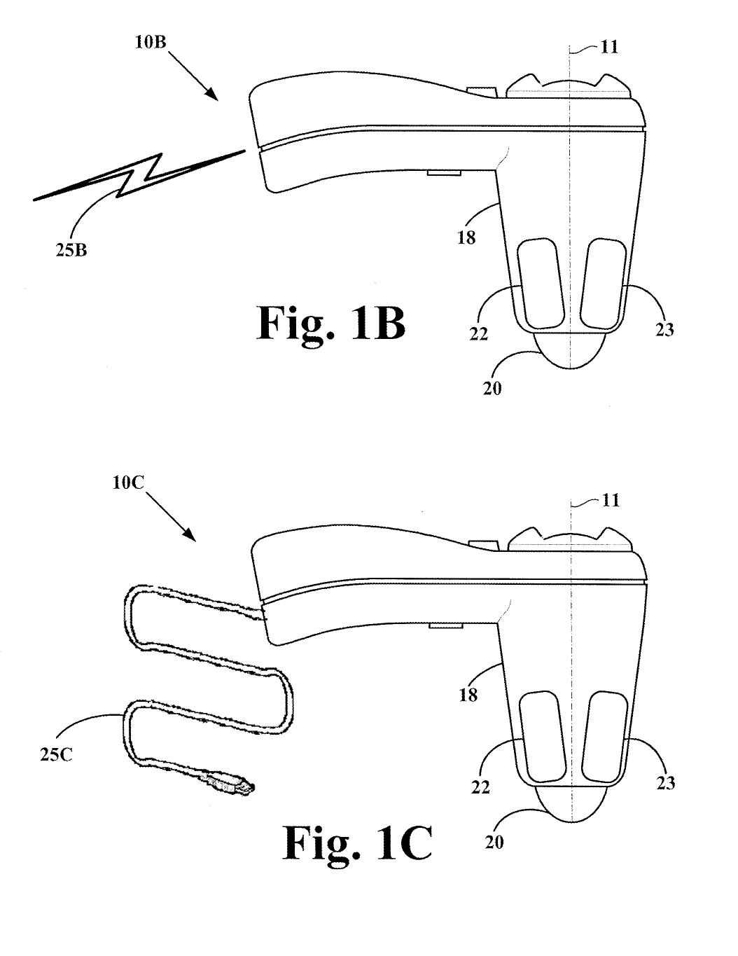

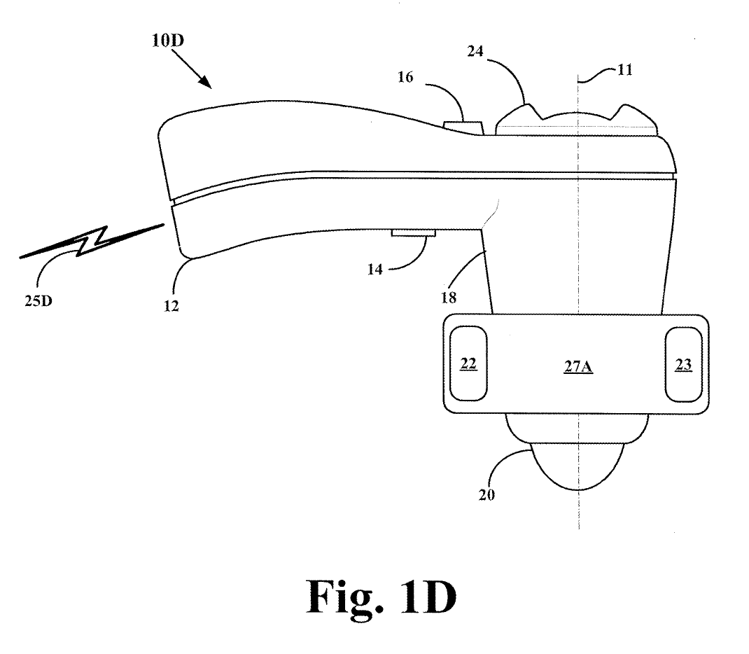

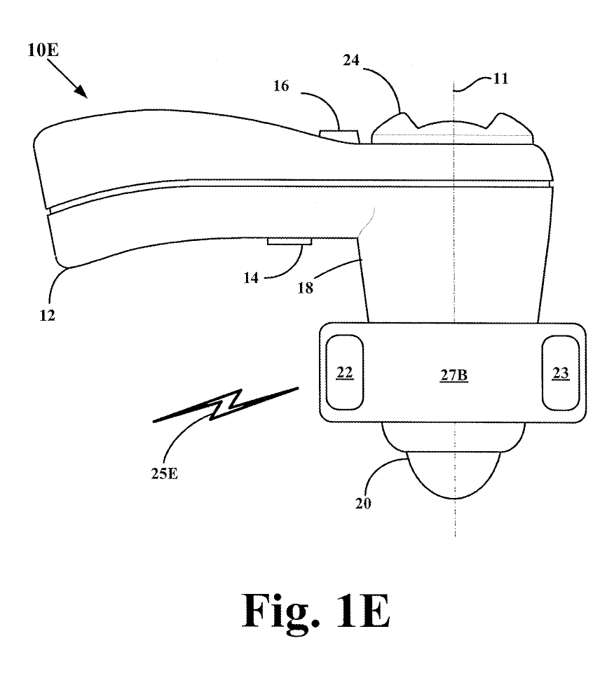

The following description and FIGS. 1 through 18 provide a thorough understanding of certain embodiments. One skilled in the art, however, will understand that the present invention may have additional embodiments, or that the present invention may be practiced without several of the details described in the following description.

According to an embodiment, FIG. 1 is a block diagrammatic view of an ultrasound system 1. System 1 includes an ultrasound unit 2 that is operable to ultrasonically scan an anatomical portion. Ultrasound unit 2 may include one or more, or a linear or non-linear array of piezoelectric elements operable to project ultrasound energy into the anatomical region, and to receive reflections from structures positioned within the anatomical region. The piezoelectric elements and/or the array may be stationary within the ultrasound unit 2, or an actuator may be provided that rotates and/or oscillates and/or otherwise moves the elements of the array so that the anatomical region may be periodically scanned by the array.

The system 1 also includes an inertial reference unit 3 that is operable to generate acceleration and angular rate information for the ultrasound unit 2. The inertial reference unit 2 may include a device that is configured to sense an acceleration associated with a directional motion of the ultrasound unit 2. The inertial reference unit 2 may also include at least one device that is operable to sense angular rate information associated with the directional motion of the ultrasound unit 2. Accordingly, a device that is configured to maintain angular position or rigidity with respect to a fixed set of reference coordinates 4 may be used. The inertial reference unit 3 may be incorporated into a structural portion of the ultrasound unit 2, or it may be a detachable accessory to the ultrasound unit 2.

Ultrasound unit 2 and inertial reference unit 3 are coupled to a processor unit 5. Processor unit 5 is configured to generate radio frequency excitation for ultrasound unit 2, and to receive signals generated by ultrasound unit 2 that result from the reflected acoustic waves. Accordingly, processor unit 5 may include a transmit/receive circuit that is coupled to respective transmitter and receiver circuits, and a suitable control circuit that permits the transmitter, receiver and the transmit/receive circuit to cooperatively insonify a desired anatomical region. The processor unit 5 may also include suitable algorithms that are configured to receive acceleration and/or angular rate information from the inertial reference unit 3, and/or to integrate the acceleration and/or angular rate information along a kinematic path of the ultrasound unit 2 to generate translational and angular position information for the ultrasound unit 2. Processor unit 5 is also operable to receive two-dimensional ultrasound information from the ultrasound unit 2 and to process information to generate a plurality of two-dimensional ultrasound images. The two-dimensional ultrasound images may be combined with the translational and/or angular position information so that a three-dimensional image of the insonified region may be generated. The processor unit 5 may also include various other devices, such as a video processor, a video memory device and a display device. Processor unit 5 may be a separate unit, such as a “mainframe” processor, or it may be incorporated into other devices, such as ultrasound unit 2. Further, it will be appreciated that FIG. 1 does not necessarily illustrate every component of the system 1. Instead, emphasis is placed upon the components that are most relevant to the following disclosed apparatus and methods.

FIG. 1A is a side elevation view of an ultrasound transceiver 10A that includes an inertial reference unit, according to an embodiment of the invention. The transceiver 10A includes a transceiver housing 18 having an outwardly extending handle 12 suitably configured to allow a user to manipulate the transceiver 10A relative to a patient. The handle 12 includes a trigger 14 that allows the user to initiate an ultrasound scan of a selected anatomical portion, and a cavity selector 16. The cavity selector 16 will be described in greater detail below. The transceiver 10A also includes a transceiver dome 20 that contacts a surface portion of the patient when the selected anatomical portion is scanned. The dome 20 generally provides an appropriate acoustical impedance match to the anatomical portion and/or permits ultrasound energy to be properly focused as it is projected into the anatomical portion. The transceiver 10A further includes one, or preferably an array of separately excitable ultrasound transducer elements (not shown in FIG. 1A) positioned within or otherwise adjacent with the housing 18. The transducer elements are suitably positioned within the housing 18 or otherwise to project ultrasound energy outwardly from the dome 20, and to permit reception of acoustic reflections generated by internal structures within the anatomical portion. The one or more array of ultrasound elements may include a one-dimensional, or a two-dimensional array of piezoelectric elements that are moved within the housing 18 by a motor. Alternately, the array may be stationary with respect to the housing 18 so that the selected anatomical region is scanned by selectively energizing the elements in the array.

Transceiver 10A includes an inertial reference unit that includes an accelerometer 22 and/or gyroscope 23 positioned preferably within or adjacent to housing 18. The accelerometer 22 is operable to sense an acceleration of the transceiver 10A, preferably relative to a coordinate system, while the gyroscope 23 is operable to sense an angular velocity of the transceiver 10A relative to the same or another coordinate system. Accordingly, the gyroscope 23 may be of conventional configuration that employs dynamic elements, or it may be an optoelectronic device, such as the known optical ring gyroscope. In one embodiment, the accelerometer 22 and the gyroscope 23 may include a commonly-packaged and/or solid-state device. One suitable commonly packaged device is the MT6 miniature inertial measurement unit, available from Omni Instruments, Incorporated, although other suitable alternatives exist. In other embodiments, the accelerometer 22 and/or the gyroscope 23 may include commonly packaged micro-electromechanical system (MEMS) devices, which are commercially available from MEMSense, Incorporated. As described in greater detail below, the accelerometer 22 and the gyroscope 23 cooperatively permit the determination of positional and/or angular changes relative to a known position that is proximate to an anatomical region of interest in the patient.

The transceiver 10A includes (or if capable at being in signal communication with) a display 24 operable to view processed results from an ultrasound scan, and/or to allow an operational interaction between the user and the transceiver 10A. For example, the display 24 may be configured to display alphanumeric data that indicates a proper and/or an optimal position of the transceiver 10A relative to the selected anatomical portion. Display 24 may be used to view two- or three-dimensional images of the selected anatomical region. Accordingly, the display 24 may be a liquid crystal display (LCD), a light emitting diode (LED) display, a cathode ray tube (CRT) display, or other suitable display devices operable to present alphanumeric data and/or graphical images to a user.

Still referring to FIG. 1A, a cavity selector 16 is operable to adjustably adapt the transmission and reception of ultrasound signals to the anatomy of a selected patient. In particular, the cavity selector 16 adapts the transceiver 10A to accommodate various anatomical details of male and female patients. For example, when the cavity selector 16 is adjusted to accommodate a male patient, the transceiver 10A is suitably configured to locate a single cavity, such as a urinary bladder in the male patient. In contrast, when the cavity selector 16 is adjusted to accommodate a female patient, the transceiver 10A is configured to image an anatomical portion having multiple cavities, such as a bodily region that includes a bladder and a uterus. Alternate embodiments of the transceiver 10A may include a cavity selector 16 configured to select a single cavity scanning mode, or a multiple cavity-scanning mode that may be used with male and/or female patients. The cavity selector 16 may thus permit a single cavity region to be imaged, or a multiple cavity region, such as a region that includes a lung and a heart to be imaged.

To scan a selected anatomical portion of a patient, the transceiver dome 20 of the transceiver 10A is positioned against a surface portion of a patient that is proximate to the anatomical portion to be scanned. The user actuates the transceiver 10A by depressing the trigger 14. In response, the transceiver 10 transmits ultrasound signals into the body, and receives corresponding return echo signals that are at least partially processed by the transceiver 10A to generate an ultrasound image of the selected anatomical portion. In a particular embodiment, the transceiver 10A transmits ultrasound signals in a range that extends from approximately about two megahertz (MHz) to approximately about ten MHz.

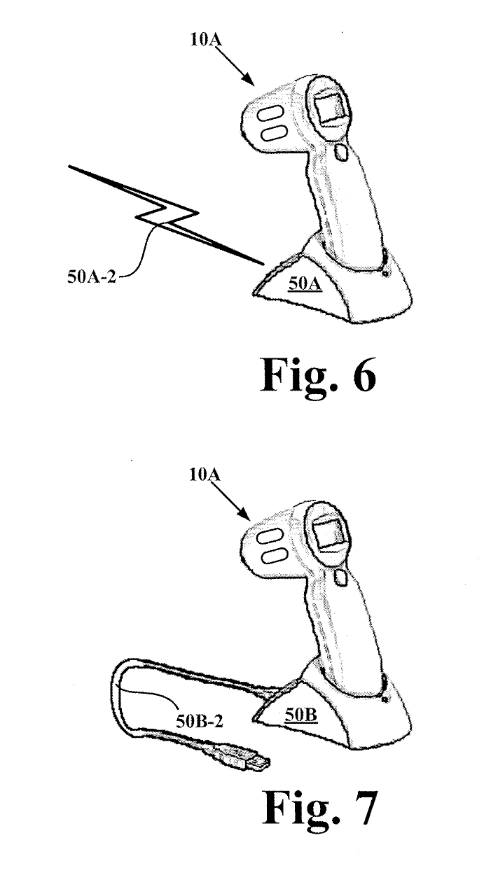

In one embodiment, the transceiver 10A is operably coupled to an ultrasound system that is configured to generate ultrasound energy at a predetermined frequency and/or pulse repetition rate and to transfer the ultrasound energy to the transceiver 10A. The system also includes a processor that is configured to process reflected ultrasound energy that is received by the transceiver 10A to produce an image of the scanned anatomical region. Accordingly, the system generally includes a viewing device, such as a cathode ray tube (CRT), a liquid crystal display (LCD), a plasma display device, or other similar display devices, that may be used to view the generated image. The system may also include one or more peripheral devices that cooperatively assist the processor to control the operation of the transceiver 10A, such a keyboard, a pointing device, or other similar devices. In still another particular embodiment, the transceiver 10A may be a self-contained device that includes a microprocessor positioned within the housing 18 and software associated with the microprocessor to operably control the transceiver 10A, and to process the reflected ultrasound energy to generate the ultrasound image. Accordingly, the display 24 is used to display the generated image and/or to view other information associated with the operation of the transceiver 10A. For example, the information may include alphanumeric data that indicates a preferred position of the transceiver 10A prior to performing a series of scans. In yet another particular embodiment, the transceiver 10A may be operably coupled to a general-purpose computer, such as a laptop or a desktop computer that includes software that at least partially controls the operation of the transceiver 10A, and also includes software to process information transferred from the transceiver 10A, so that an image of the scanned anatomical region may be generated. The transceiver 10A may also be optionally equipped with electrical contacts to make communication with accessory devices as discussed in FIGS. 6 and 7 below.

Although transceiver 10A of FIG. 1A may be used in any of the foregoing embodiments, other transceivers may also be used. For example, the transceiver may lack one or more features of the transceiver 10A. For example, a suitable transceiver need not be a manually portable device, and/or need not have a top-mounted display, and/or may selectively lack other features or exhibit further differences.

FIG. 1B is a side elevation view of an ultrasound transceiver 10B that includes an inertial reference unit, according to another embodiment of the invention. Many of the details of the ultrasound transceiver 10B have been discussed in connection with FIG. 1A, and in the interest of brevity, will not be repeated. The transceiver 10B is optionally configured to communicate signals wirelessly to other external devices. For example, wireless signals 25B may include imaging data and/or positional information acquired by the transceiver 10B that is transferred from the transceiver 10B to an external processing device (not shown in FIG. 1B) that provides additional processing of the imaging data.

FIG. 1C is a side elevation view of an ultrasound transceiver 10C that includes an inertial reference unit, according to still yet another embodiment of the invention. In this embodiment, the transceiver 10C is configured to communicate signals through an interface cable 25C to other external devices. For example, the signals communicated on the interface cable 25C may include imaging data and/or positional information acquired by the transceiver 10B that is transferred from the transceiver 10B to an external processing device (not shown in FIG. 1C) that provides additional processing of the imaging data. The interface cable 25C may be configured to communicate data in accordance with any known or future data interface protocol. Consequently, the interface cable 25C may be configured to communicate data using the known Universal Serial Bus protocol (USB), or using other known protocols, such as FIREWIRE, serial or even parallel port-configured cables. Alternatively, the interface cable 25C may be a fiber optic cable that is operable to convey light-based signals.

FIG. 1D is a side elevation view of an ultrasound transceiver 100 according to still another embodiment of the invention. The transceiver 10D includes an inertial reference unit 27A that is demountably coupled to one of the housing 18 or handle 12, and that includes a positional sensing device such as the accelerometer 22 and/or an angular sensing device, such as the gyroscope 23. The inertial reference unit as illustrated may have a collar configuration that circumscribes the housing 18. Other demountable or detachable configurations are possible, for example, a slide-on tube detachably attachable to the handle 12. The demountably couplable inertial reference unit 27A is configured to be mounted on an ultrasound transceiver that does not have an inertial reference sensing capability. A wireless signal 25D is emitted from the transceiver 10D that includes acceleration and/or rate information generated by the accelerometer 22 and/or the gyroscope 23. The foregoing accelerometer and rate information are routed from the inertial reference unit 27A in the transceiver 10D through corresponding electrical contacts between inertial reference unit 27A and the housing 18. Alternate embodiments of the transceiver 10D include non-wireless signals conveyed through electrical cables and/or fiber optics, such as, for example, those previously described.

FIG. 1E is side elevation view of an ultrasound transceiver 10E according to another embodiment of the invention. The transceiver 10E also includes an inertial reference unit 27B that is detachably or demountably couplable to the housing 18. The unit 27B also optionally includes a wireless transmitter (not shown), and/or the accelerometer 22 and/or gyroscope 23. The transceiver 10E is shown with the detachably demountably couplable unit 27B in a collar configuration that detachably demountably circumscribes the housing 18. The collar 27B similarly snaps onto a non-inertial reference transceiver and converts it to an inertial reference transceiver 10E that suitably operates similar to transceiver 10B of FIG. 1B except that a wireless signal 25E emanates from the collar 27B. The wireless signal 25E contains the positional information of the accelerometer 22 and/or gyroscope 23. Other detachable or demountable configurations of the inertial reference unit 27B are possible, for example, a slide-on tube demountably attachable to the handle 12. Alternate embodiments of the transceiver 10E include non-wireless signals conveyed through electrical cables and fiber optics previously described.

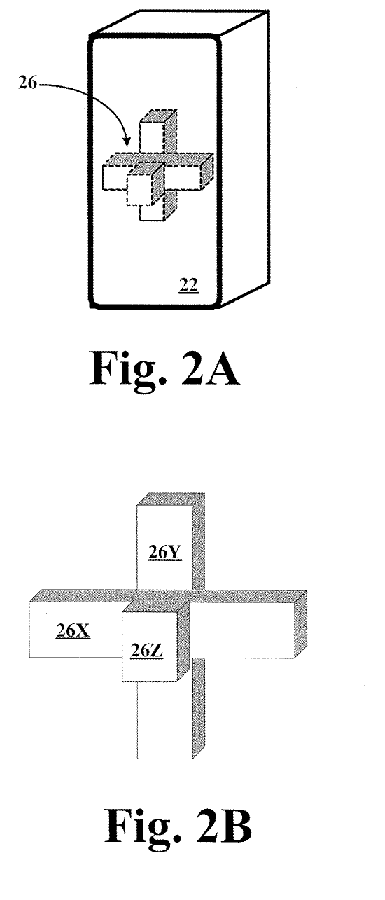

FIG. 2A is a schematic illustration of the accelerometer of the transceivers 10A-10E of FIGS. 1A-1E, respectively. An accelerometer array 26 may be internally disposed within the accelerometer 22. The array 26 is shown by dashed lines in FIG. 2A, and includes elements that are generally oriented in mutually orthogonal directions. The accelerometer 26 may be oriented in any selected orientation with respect to the transceivers 10A, 10B and 10C.

FIG. 2B is an expansion of the schematic illustration of FIG. 2A. The accelerometer array 26 includes an X-axis, Y-axis, and Z-axis oriented elements 26X, 26Y, and 26Z, respectively. The accelerometer elements 26X, 26Y, and 26Z are presented as a stacked array, although other configurations are possible. For example, a planar configuration may also be used. In either case, the X-axis, Y-axis, and Z-axis accelerometer elements 28X, 28Y, and 28Z generate electrical signals that proportional to or otherwise indicative of accelerations along the respective X, Y, and Z-axes.

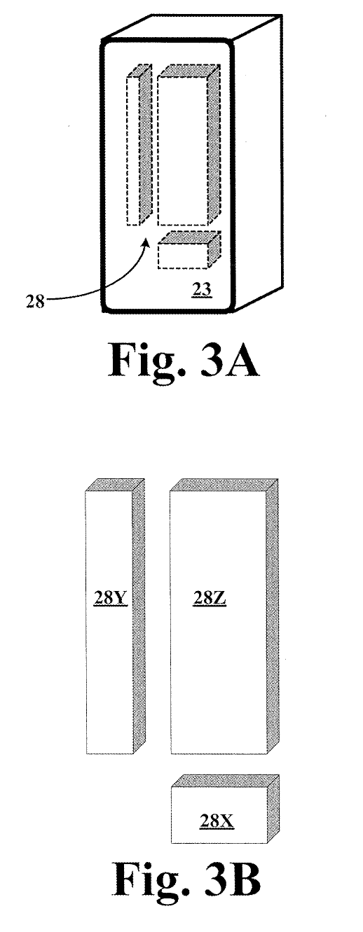

FIG. 3A is a schematic illustration of the gyroscope of the transceivers 10A-10E of FIGS. 1A-1E, respectively. A gyroscope array 28 may be internally disposed within the gyroscope 23. The array 28 is shown by dashed lines in FIG. 3A, and includes elements that are generally oriented in mutually orthogonal directions. The gyroscope 23 may be oriented in any selected orientation with respect to the transceivers 10A-10E.

FIG. 3B is an expansion of the schematic illustration of FIG. 3A. The gyroscope array 28 generally includes an X-axis, Y-axis, and Z-axis oriented elements 28X, 28Y, and 28Z, respectively. The elements 26X, 26Y, and 26Z are operable to sense motions about X, Y and Z axes, respectively, and generate electrical signals that are proportional to motions about the respective X, Y, and Z-axes.

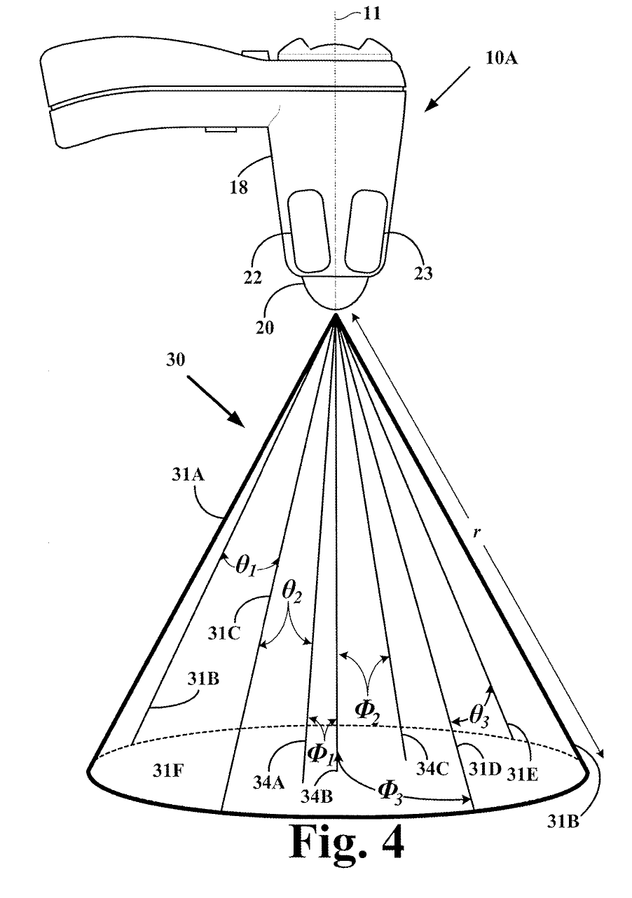

FIG. 4 is a graphical representation of a plurality of three dimensional (3D) distributed scan lines emanating from a transceiver that cooperatively forms a scan cone 30. Each of the scan lines have a length r that projects outwardly from the transceivers 10A-10E of FIGS. 1A-1E. As illustrated the transceiver 10A emits 3D-distributed scan lines within the scan cone 30 that are one-dimensional ultrasound A-lines. The other transceiver embodiments 10B-10E may also be configured to emit 3D-distributed scan lines. Taken as an aggregate, these 3D-distributed A-lines define the conical shape of the scan cone 30. The ultrasound scan cone 30 extends outwardly from the dome 20 of the transceiver 10A, 10B and 10C centered about an axis line 11. The 3D-distributed scan lines of the scan cone 30 include a plurality of internal and peripheral scan lines that are distributed within a volume defined by a perimeter of the scan cone 30. Accordingly, the peripheral scan lines 31A-31F define an outer surface of the scan cone 30, while the internal scan lines 34A-34C are distributed between the respective peripheral scan lines 31A-31F. Scan line 34B is generally collinear with the axis 11, and the scan cone 30 is generally and coaxially centered on the axis line 11.

The locations of the internal and peripheral scan lines may be further defined by an angular spacing from the center scan line 34B and between internal and peripheral scan lines. The angular spacing between scan line 34B and peripheral or internal scan lines are designated by angle Φ and angular spacings between internal or peripheral scan lines are designated by angle Ø. The angles Φ₁, Φ₂ and Φ₃ respectively define the angular spacings from scan line 34B to scan lines 34A, 34C, and 31D. Similarly, angles Ø₁, Ø₂, and Ø₃ respectively define the angular spacings between scan line 31B and 31C, 31C and 34A, and 31D and 31E.

With continued reference to FIG. 4, the plurality of peripheral scan lines 31A-E and the plurality of internal scan lines 34A-D are three dimensionally distributed A-lines (scan lines) that are not necessarily confined within a scan plane, but instead may sweep throughout the internal regions and along the periphery of the scan cone 30. Thus, a given point within the scan cone 30 may be identified by the coordinates r, Φ, and Ø whose values generally vary. The number and location of the internal scan lines emanating from the transceivers 10A-10E may thus be distributed within the scan cone 30 at different positional coordinates as required to sufficiently visualize structures or images within a region of interest (ROI) in a patient. The angular movement of the ultrasound transducer within the transceiver 10A-10E may be mechanically effected, and/or it may be electronically generated. In any case, the number of lines and the length of the lines may be uniform or otherwise vary, so that angle (d sweeps through angles approximately between −60° between scan line 34B and 31A, and +600 between scan line 34B and 31B. Thus angle ( ) in this example presents a total arc of approximately 120°. In one embodiment, the transceiver 10A, 10B and 10C is configured to generate a plurality of 3D-distributed scan lines within the scan cone 30 having a length r of approximately 18 to 20 centimeters (cm).

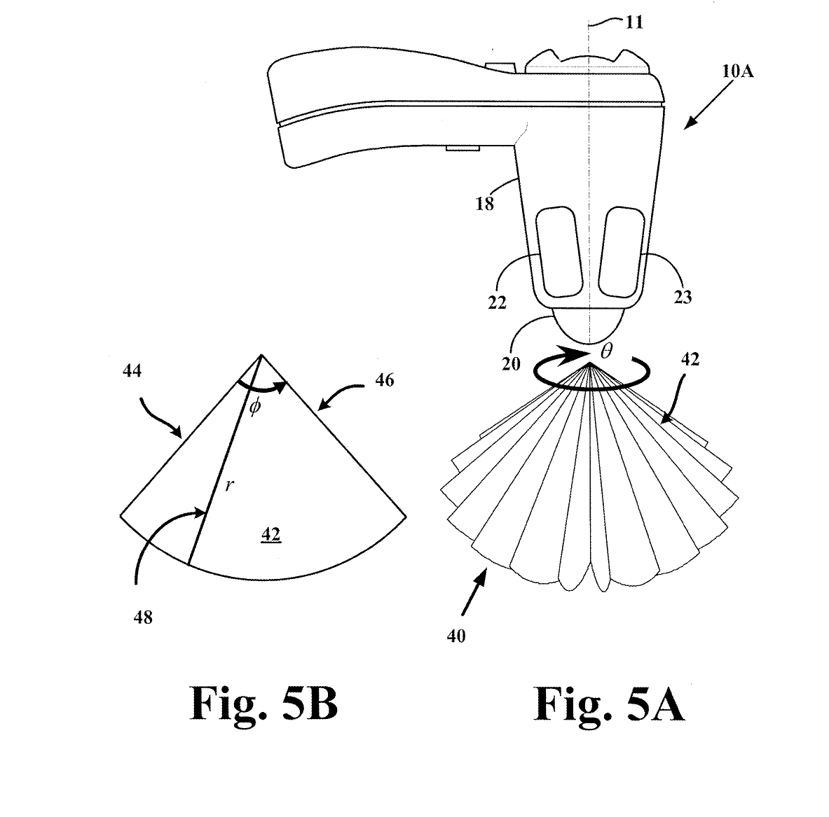

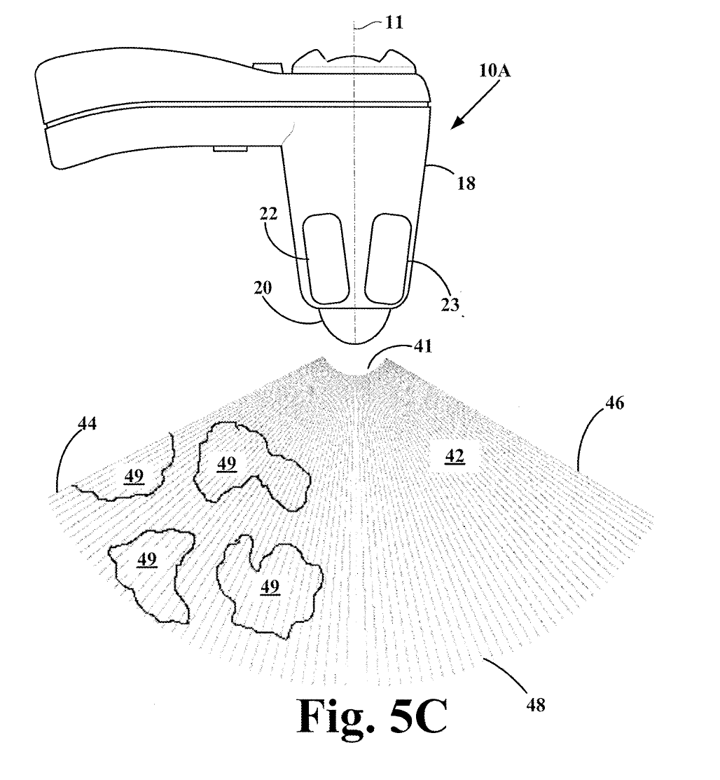

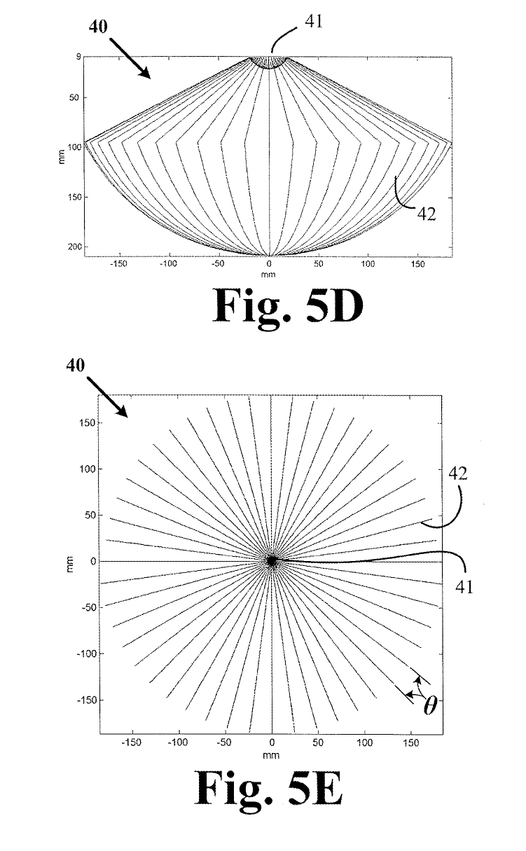

FIG. 5A is a graphical representation of a plurality of scan planes that form a three-dimensional (3D) array having a substantially conical shape. An ultrasound scan cone 40 formed by a rotational array of two-dimensional scan planes 42 projects outwardly from the dome 20 of the transceivers 10A. The other transceiver embodiments 10B-10E may also be configured to develop a scan cone 40 formed by a rotational array of two-dimensional scan planes 42. The plurality of scan planes 40 are oriented about an axis 11 extending through the transceivers 10A-10E. One or more, or preferably each of the scan planes 42 are positioned about the axis 11, preferably, but not necessarily at a predetermined angular position θ. The scan planes 42 are mutually spaced apart by angles θ₁ and θ₂. Correspondingly, the scan lines within each of the scan planes 42 are spaced apart by angles φ₁ and φ₂. Although the angles θ₁ and θ₂ are depicted as approximately equal, it is understood that the angles θ₁ and θ₂ may have different values. Similarly, although the angles φ₁ and φ₂ are shown as approximately equal, the angles φ₁ and φ₂ may also have different angles. Other scan cone configurations are possible. For example, a wedge-shaped scan cone, or other similar shapes may be generated by the transceiver 10A, 10B and 10C.

FIG. 5B is a graphical representation of a scan plane 42. The scan plane 42 includes the peripheral scan lines 44 and 46, and an internal scan line 48 having a length r that extends outwardly from the transceivers 10A-10E. Thus, a selected point along the peripheral scan lines 44 and 46 and the internal scan line 48 may be defined with reference to the distance r and angular coordinate values φ and θ. The length r preferably extends to approximately 18 to 20 centimeters (cm), although any length is possible. Particular embodiments include approximately seventy-seven scan lines 48 that extend outwardly from the dome 20, although any number of scan lines is possible.

FIG. 5C a graphical representation of a plurality of scan lines emanating from a hand-held ultrasound transceiver forming a single scan plane 42 extending through a cross-section of an internal bodily organ. The number and location of the internal scan lines emanating from the transceivers 10A-10E within a given scan plane 42 may thus be distributed at different positional coordinates about the axis line 11 as required to sufficiently visualize structures or images within the scan plane 42. As shown, four portions of an off-centered region-of-interest (ROI) are exhibited as irregular regions 49. Three portions are viewable within the scan plane 42 in totality, and one is truncated by the peripheral scan line 44.

As described above, the angular movement of the transducer may be mechanically effected and/or it may be electronically or otherwise generated. In either case, the number of lines 48 and the length of the lines may vary, so that the tilt angle φ sweeps through angles approximately between −60° and +600 for a total arc of approximately 120°. In one particular embodiment, the transceiver 10 is configured to generate approximately about seventy-seven scan lines between the first limiting scan line 44 and a second limiting scan line 46. In another particular embodiment, each of the scan lines has a length of approximately about 18 to 20 centimeters (cm). The angular separation between adjacent scan lines 48 (FIG. 5B) may be uniform or non-uniform. For example, and in another particular embodiment, the angular separation φ₁ and φ₂ (as shown in FIG. 5C) may be about 1.5°. Alternately, and in another particular embodiment, the angular separation φ₁ and φ₂ may be a sequence wherein adjacent angles are ordered to include angles of 1.5°, 6.8°, 15.5°, 7.2°, and so on, where a 1.5° separation is between a first scan line and a second scan line, a 6.8° separation is between the second scan line and a third scan line, a 15.5° separation is between the third scan line and a fourth scan line, a 7.20 separation is between the fourth scan line and a fifth scan line, and so on. The angular separation between adjacent scan lines may also be a combination of uniform and non-uniform angular spacings, for example, a sequence of angles may be ordered to include 1.5°, 1.5°, 1.5°, 7.2°, 14.3°, 20.2°, 8.0°, 8.0°, 8.0°, 4.3°, 7.8°, and so on.

FIG. 5D is an isometric view of an ultrasound scan cone that projects outwardly from the transceivers of FIGS. 1A-E. Three-dimensional mages of a region of interest are presented within a scan cone 40 that comprises a plurality of 2D images formed in an array of scan planes 42. A dome cutout 41 that is the complementary to the dome 20 of the transceivers 10A-10E is shown at the top of the scan cone 40.

FIG. 5E is a top plan view of the scan cone 40 of FIG. 5D. The arrangement of the scan planes 42 is shown symmetrically distributed or radiating from the cutout 41 and separated by an angle θ. The angle θ may vary so that the angular spacings may result in the scan cone 40 having an array of non-symmetrically distributed scan planes.

FIG. 6 and FIG. 7 are respective isometric views of a transceiver 10A having an inertial reference unit, according to an embodiment of the invention. With reference to FIG. 6, the transceiver 10A is received by a support cradle 50A. The cradle 50A is structured to perform various support functions that assist the transceiver 10A. For example, the support cradle 50A may be configured to exchange wireless signals 50A-2 with other devices, such as an external processor. The support cradle 50A may also include a battery charger that is operable to charge an internal battery that is positioned within the transceiver 10A. With reference now to FIG. 7, the transceiver 10B is received by a support cradle 50B that includes an interface unit that is operable to receive ultrasound and/or positional information from the transceiver 10A, and optionally to format the information according to a suitable data interface protocol. Accordingly, the cradle 50 includes an interface cable 50B-2 that is configured to exchange the formatted information with an external device.

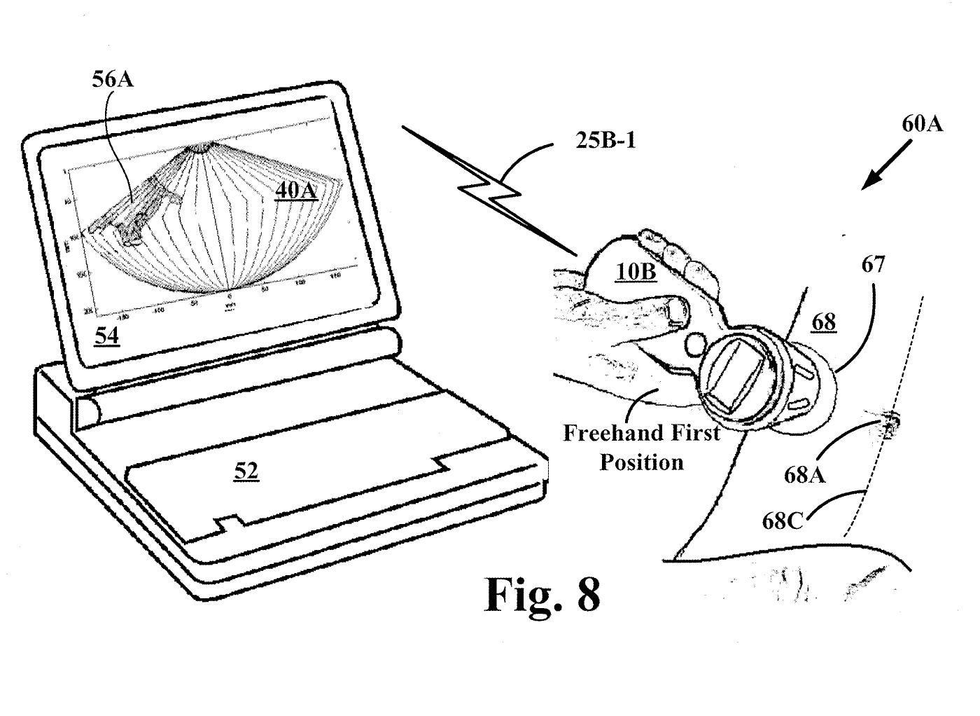

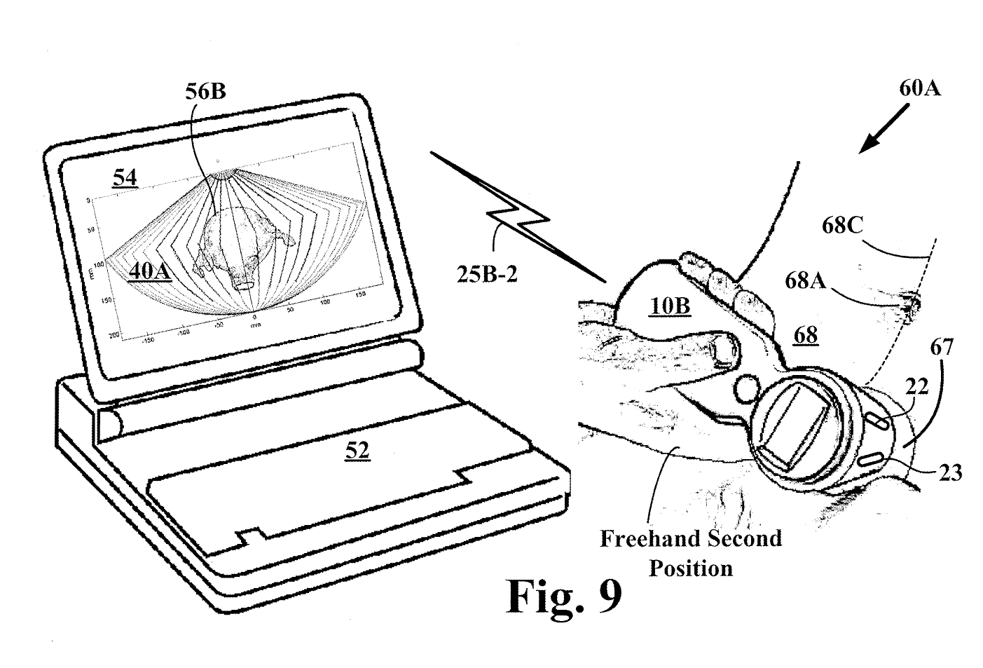

FIG. 8 is an isometric view of an inertial ultrasound imaging system 60A according to another embodiment of the invention. The system 60A includes the transceiver 10B of FIG. 1B, although the transceiver 10C of FIG. 1C may also be used without significant modification. The system 60A also includes a personal computing device 52 that is configured to wirelessly exchange information with the transceiver 10B. Any means of information exchange can be employed when the transceiver 10C is used. In operation, the transceiver 10B is applied to a side abdominal region of a patient 68. The transceiver 10B is placed off-center from a centerline 68C of the patient 68 to obtain, for example a trans-abdominal image of a uterine organ in a female patient. The transceiver 10B may contact the patient 68 through a pad 67 that includes an acoustic coupling gel that is placed on the patient 68 substantially left of the umbilicus 68A and centerline 68C. Alternatively, an acoustic coupling gel may be applied to the skin of the patient 68. The pad 67 advantageously minimizes ultrasound attenuation between the patient 68 and the transceiver 10B by maximizing sound conduction from the transceiver 10B into the patient 68.

Wireless signals 25B-1 contain echo information that is conveyed to and processed by the image processing algorithm in the personal computer device 52. A scan cone 40A displays an internal organ as partial image 56A on a computer display 54. The image 56A is significantly truncated and off-centered relative to a middle portion of the scan cone 40A due to the positioning of the transceiver 10B.

As shown in FIG. 8, the trans-abdominally acquired image is initially obtained during a targeting phase of the imaging. The transceiver 10B is operated in a two-dimensional continuous acquisition mode. In the two-dimensional continuous mode, data is continuously acquired and presented as a scan plane image as previously shown and described. The data thus acquired may be viewed on a display device, such as the display 54, coupled to the transceiver 10B while an operator physically translates the transceiver 10B across the abdominal region of the patient. When it is desired to acquire data, the operator may acquire data by depressing the trigger 14 of the transceiver 10B to acquire real-time imaging that is presented to the operator on the display device. If the initial location of the transceiver is significantly off-center, in this case only a portion of the organ 56 is visible in the scan plane 40A.

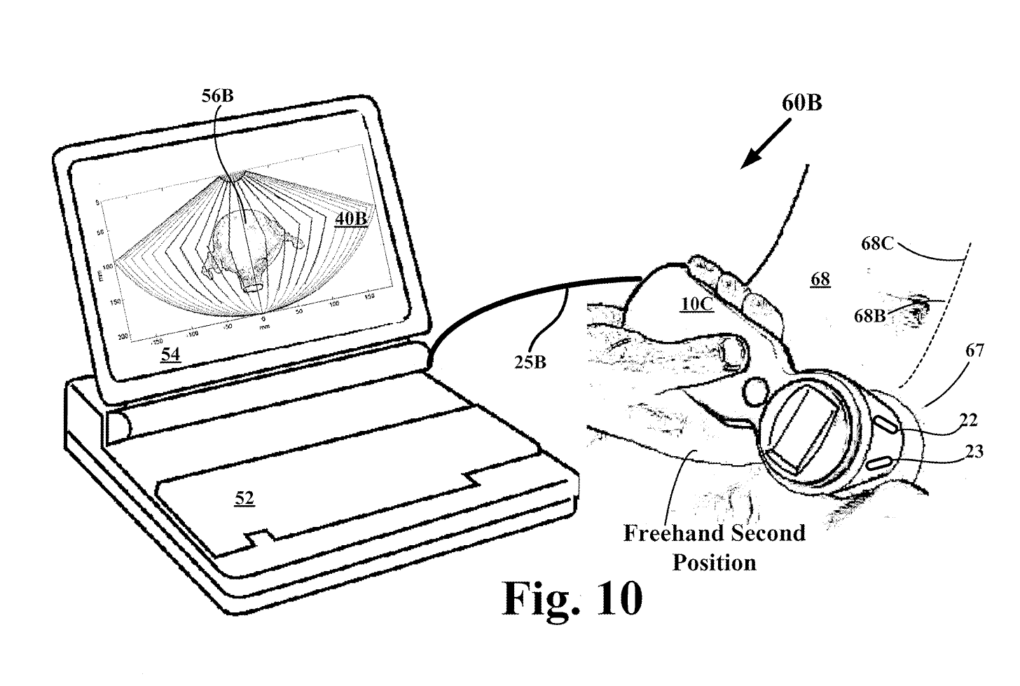

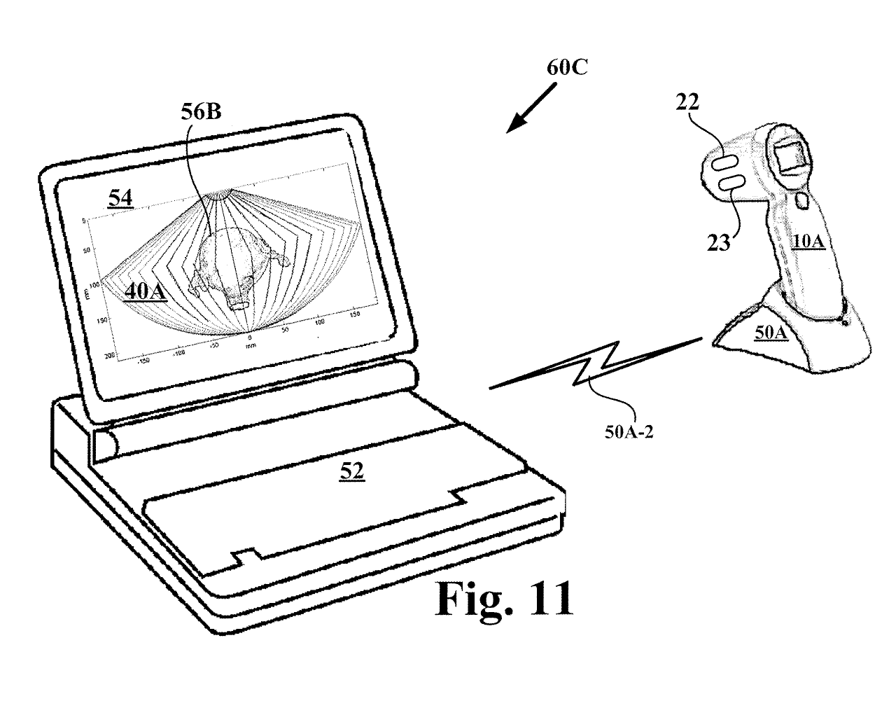

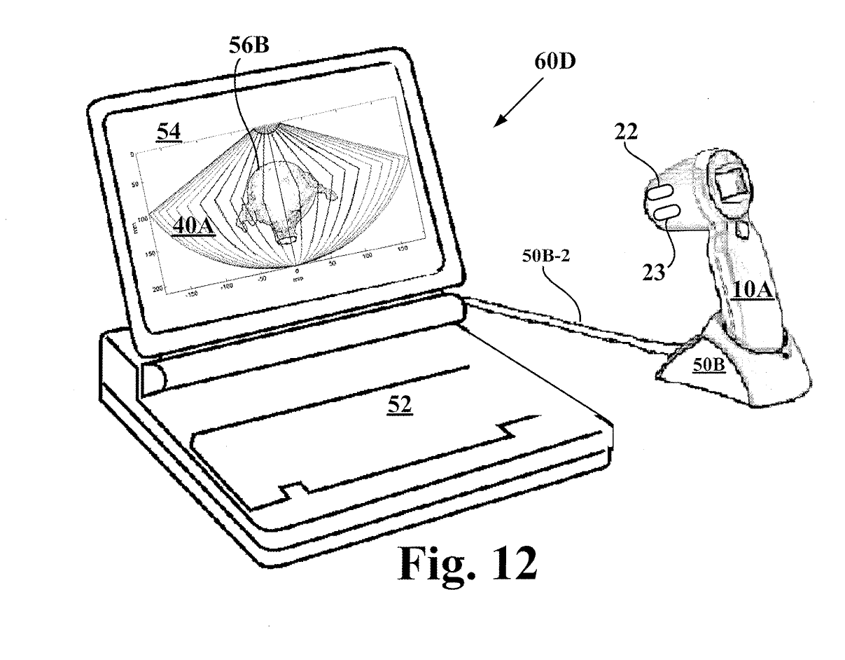

FIG. 9 is an isometric view of an inertial ultrasound imaging system 60A according to another embodiment of the invention. The system 60A includes the transceiver of FIG. 1B and is applied to a center abdominal region of a patient. The transceiver 10B may be freehand translated to a position beneath the umbilicus 68A on the centerline 68C of the patient 68. Wireless signals 25B-2 having information from the transceiver 10B is communicated to the personal computer device 52. The inertial reference unit positioned within the transceiver 10B senses positional changes for the transceiver 10B relative to a reference coordinate system. Information from the inertial reference unit, as described in greater detail below, permits updated real-time scan cone image acquisition, so that a scan cone 40B having a complete image of the organ 56B can be obtained. Still other embodiments are within the scope of the present invention. For example, the transceiver 10C of FIG. 1C may also be used in the system 60A, as shown in FIG. 10. The transceiver 10A and the support cradle 50A shown in FIG. 6 as well as the transceiver 10A and the support cradle 50B may also be used, as shown in FIG. 11 and FIG. 12, respectively.

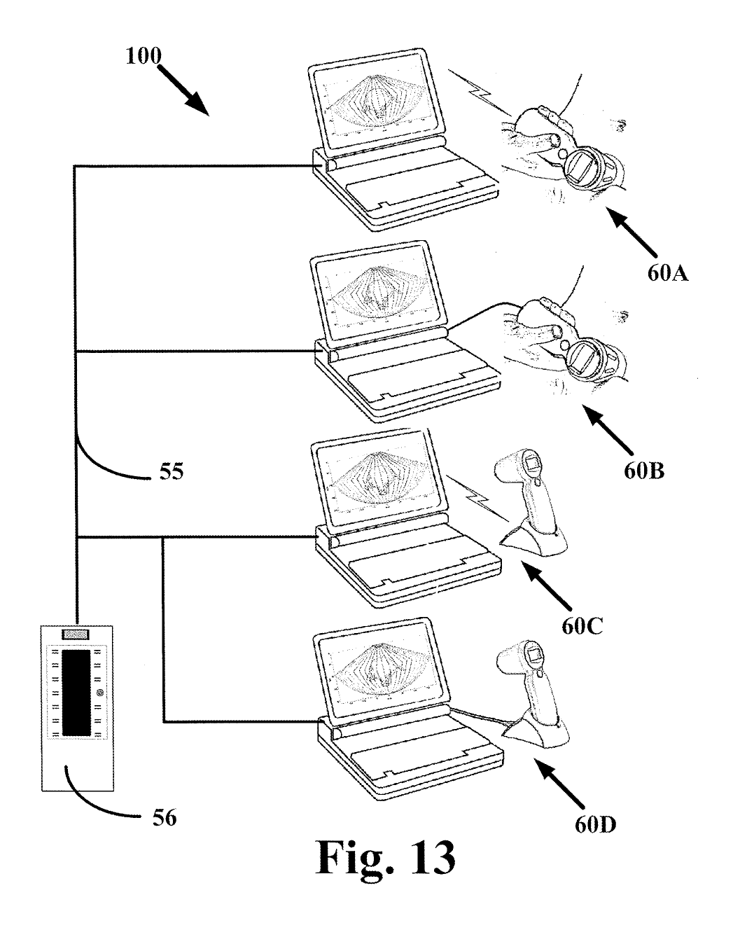

FIG. 13 is a partial isometric view of an ultrasound system 100 according to another embodiment of the invention. The system 100 includes one or more personal computer devices 52 that are coupled to a server 56 by a communications system 55. The devices 52 are, in turn, coupled to one or more ultrasound transceivers, for examples the systems 60A-60D. The server 56 may be operable to provide additional processing of ultrasound information, or it may be coupled to still other servers (not shown in FIG. 13) and devices, for examples transceivers 10D and 10E having snap on collars 27A and 27B respectively.

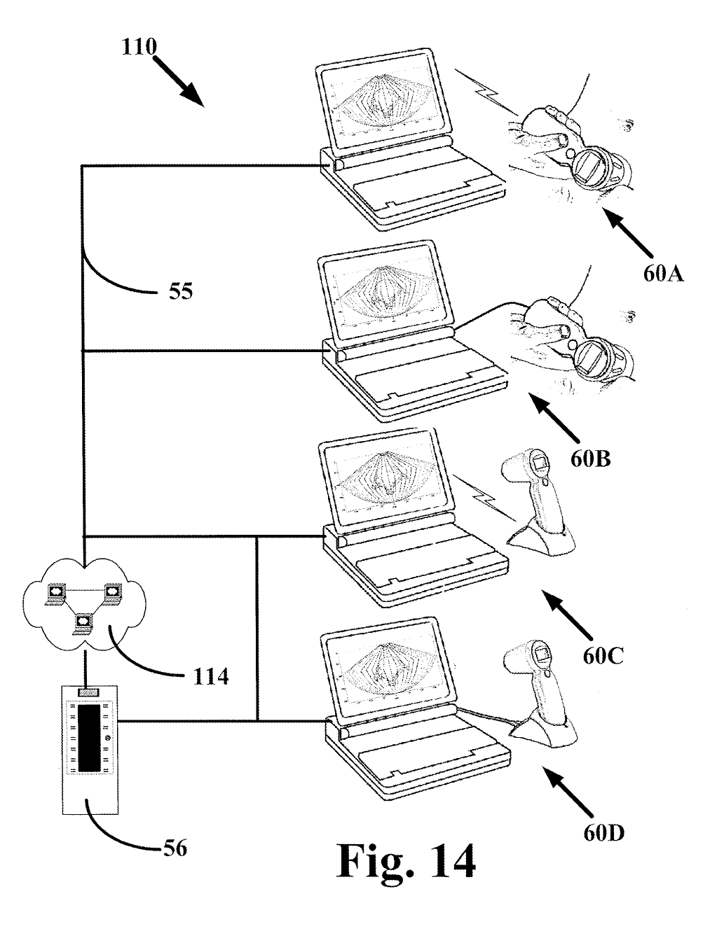

FIG. 14 is a schematic illustration of the Internet in communication with the inertial ultrasound imaging systems of FIGS. 9-12. An Internet system 110 is coupled or otherwise in communication with the systems 60A-60D. The system 110 may also be in communication with the transceivers 10D and 10E.

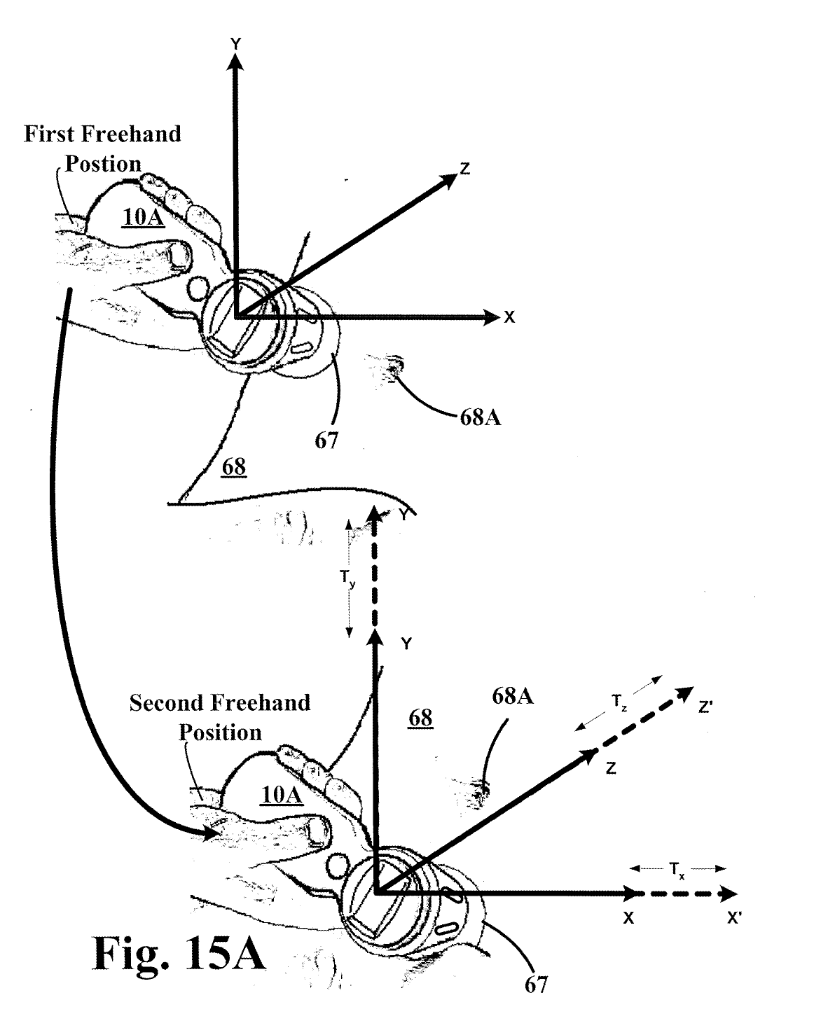

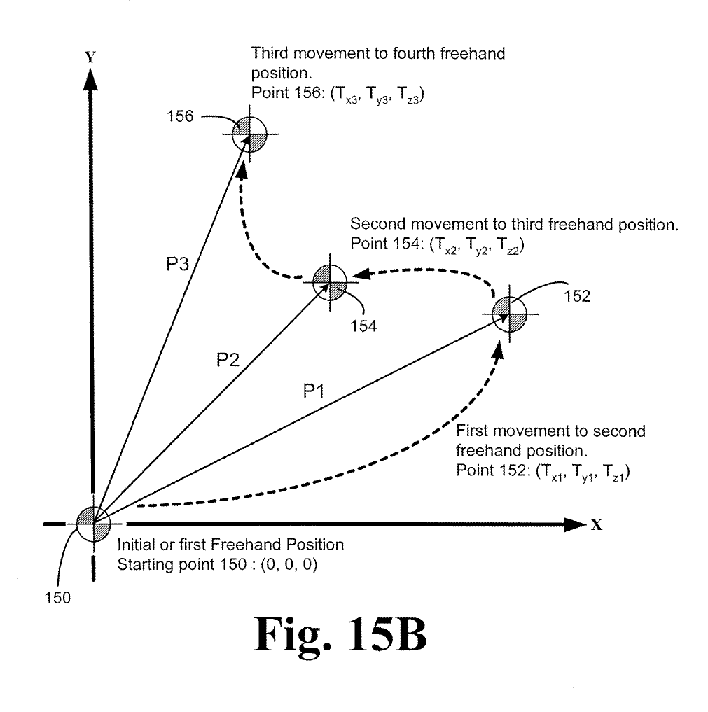

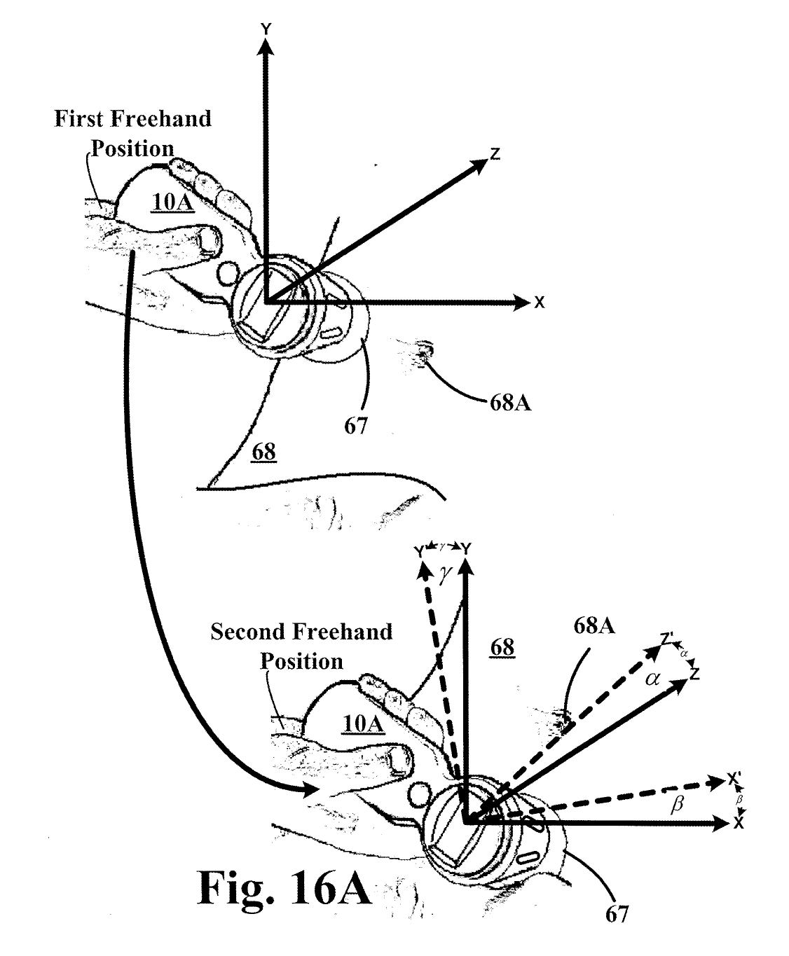

FIG. 15A is a schematic illustration of inertial reference coordinates superimposed over a transceiver experiencing translation changes between two transceiver locations regions. The transceiver locations provide different ultrasound probe views of a patient's ROI via the transceivers 10A-10E. Referring now to transceiver 10 A, but not excluding the other transceivers 10B-10E embodiments previously described, freehand translations of the transceiver 10A will cause changes in at least one Cartesian coordinate axis value. Changes of either X, Y, or Z locations, or possibly any combination thereof depending on the user's repositioning of the transceiver 10A and whether or not there is only a single or multiple axis translation from the first to the second freehand positions can occur. As shown in this illustration, translation only is shown in that there is an absence of rotation or tilt of the transceiver 10A. The first freehand position Cartesian axes and designated as X-Y-Z and the second Cartesian axes are designated as X′-Y′-Z′. The respective differences due to translation for each axis are designated as translation values Tₓ, T_{y}, and T_{z}.

FIG. 15B further describes schematically the translation movements from an initial or first freehand position 150 overlaid on an X-Y Cartesian plot. The dashed curved arrows indicate the freehand movement path to positional points from the initial freehand position 150. As earlier described, the transceiver 10A may be positioned in various positions relative to a patient, so that different two-dimensional views of a desired anatomical region of interest may be generated. Accordingly, the transceiver 10A (as shown in FIG. 1A) may be positioned at the first transceiver or initial position 150, whereupon the inertial reference unit (as shown in FIG. 1) is aligned, so that the position 150 may be used as an origin for the various freehand positions. As illustrated, the initial position point 150 is located at the X-Y-Z axes origin and may be conveniently defined by a component set of (0, 0, 0). All subsequent positional movements may then be referenced to the initial position 150. The first transceiver position 150 may include a positional location that is proximate to a desired anatomical portion of the patient, or it may include a positional location that is spaced apart from the patient. In either case, the transceiver 10A may be moved to still other locations, such as a second transceiver position 152, a third transceiver position 154, and a fourth transceiver position 156, although though other positional locations relative to the first transceiver position 150 may also be used. As illustrated, transceiver locations 152, 154 and 156 reside in the first Cartesian quadrant, though any transceiver location may be within other Cartesian quadrants or occupy a Cartesian axis. Respective coordinates for each of the vectors P1, P2, and P3 respectively extending to the second position 152, the third position 154 and the fourth position 156 and may be readily expressed as vector components in the form of Tₓᵢ, T_{yi}, and T_{zi} where i corresponds to a selected one of the vectors. Accordingly, vector P1 from the initial component set to the second position point 152 is defined by component set (Tₓ₁, T_{y1}, and T_{z1}) derived from positional information obtained from the accelerometer 22. Similarly, movement to the third positional point 154 is described by vector P2 having a component set (Tₓ₂, T_{y2}, and T_{z2}). Thereafter, movement to the fourth positional point 156 is described by vector P3 having a component set (Tₓ₃, T_{y3}, and T_{z3}).

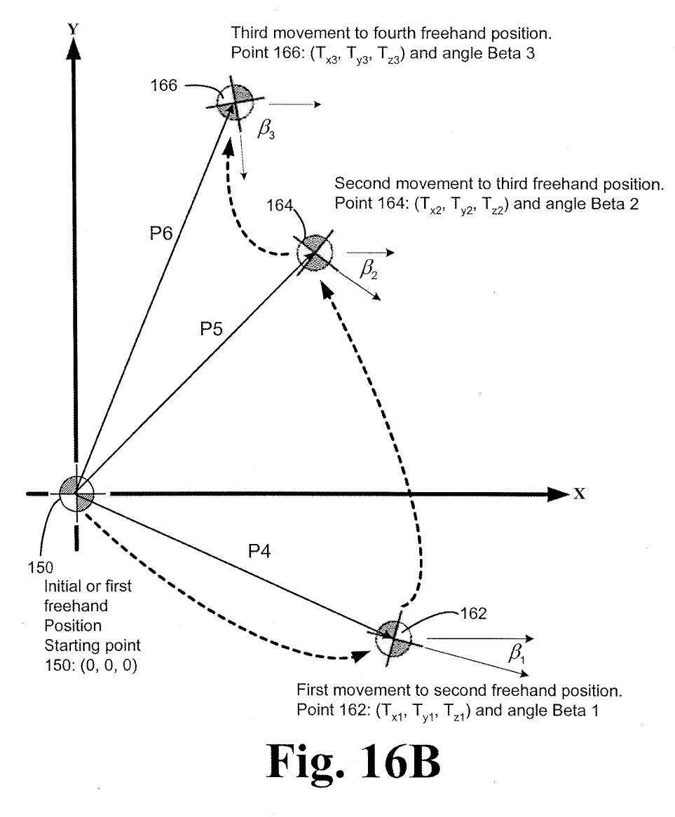

FIG. 16A is a schematic illustration of inertial reference coordinates superimposed over a transceiver experiencing rotation and tilt changes between two transceiver locations regions. The transceiver locations provide different translational and/or rotational ultrasound probe views of a patient's ROI. Freehand translations of the transceiver 10A will cause changes in at least one Cartesian coordinate axis value previously described, and whether or not there is any tilt or rotation of the transceiver 10A between an initial and succeeding freehand positioning. Thus a change in location of a given point P of an ROI can be defined in Cartesian terms with angular values. By way of example, a solid lined X-Y-Z Cartesian axis overlaid upon the transceiver 20 in the first freehand position is compared to a dashed lined X′-Y′-Z′Cartesian axis overlaid upon the transceiver 20 in the second freehand position. Changes in translation values of the X and Y-axes are shown as angular displacements γ and β, respectively. Similarly, changes in rotation about the Z-axis are angle values α. Thus changes between X of the first freehand position and X′ of the second freehand position are defined by angle γ, Y of the first freehand position and Y′ of the second freehand position are defined by angle β, and Z of the first freehand position and Z′ of the second freehand position are defined by angle α. The accelerometer array 26 and the gyroscope array 28 cooperatively determined the changes in angular displacements α, β, and γ through their respective X, Y, and Z-axis specific accelerometers and gyroscopes as illustrated in FIGS. 2B and 3B.

FIG. 16B is a schematic illustration that will be used to further describe the method of FIG. 16A involving a series of translation and rotation movements from an initial freehand position. The angular positions of the transceiver 10A may also be determined that are relative to the first transceiver position 150. Beginning with the inertial reference unit (as shown in FIG. 1) at position 150, a series of motions having translation and rotation results in a second transceiver position 162, a third transceiver position 164, and a fourth transceiver position 166. The second transceiver position is located in the fourth Cartesian quadrant and the third and fourth transceiver positions 164 and 166 are located within the first Cartesian quadrant. Respective coordinates for each of the vectors P4, P5, and P6 extending to the second position 162, the third position 164 and the fourth position 166, may respectively be readily defined as translation point sets in the form of Tₓᵢ, T_{yi}, and T_{zi} and angle β. For example, the second transceiver position 162 may include a first rotational angle β₁, while the third transceiver position 164 and the fourth transceiver position 166 include second and third rotational angles, β₂ and β₃, respectively. Accordingly, vector P4 from the initial position 150 to the second position point 162 is defined by point set (Tₓ₁, T_{y1}, and T_{z1}) derived from positional information obtained from the accelerometer 22 and angle β₁ positional information obtained from the gyroscope 23. Similarly, movement to the third positional point 154B is described by vector P2B having a point set (Tₓ₂, T_{y2}, and T_{z2}) and angle β₂. Thereafter, movement to the fourth positional point 156B is described by vector P3B having a point set (Tₓ₃, T_{y3}, and T_{z3}) and angle β₃. Although FIG. 16B shows the first rotational angle β₁, the second rotational angle β₂, and the third rotational angle β₃ positioned in one plane, it is understood that rotational angles also generally exist in other rotational planes.

The positional coordinates and angles that are determined relative to the first position 150 may be used to combine the two-dimensional images determined at each of the positions into a three-dimensional ultrasound image. Although FIGS. 15A and 15B describes a translational movement of the transceiver 10A relative to the first position 150, and FIGS. 16A and 16B describes rotations of the transceiver 10A relative to the position 150, it is understood that successive movements of the transceiver 10A generally include both translational movements and rotations of the transceiver 10A.

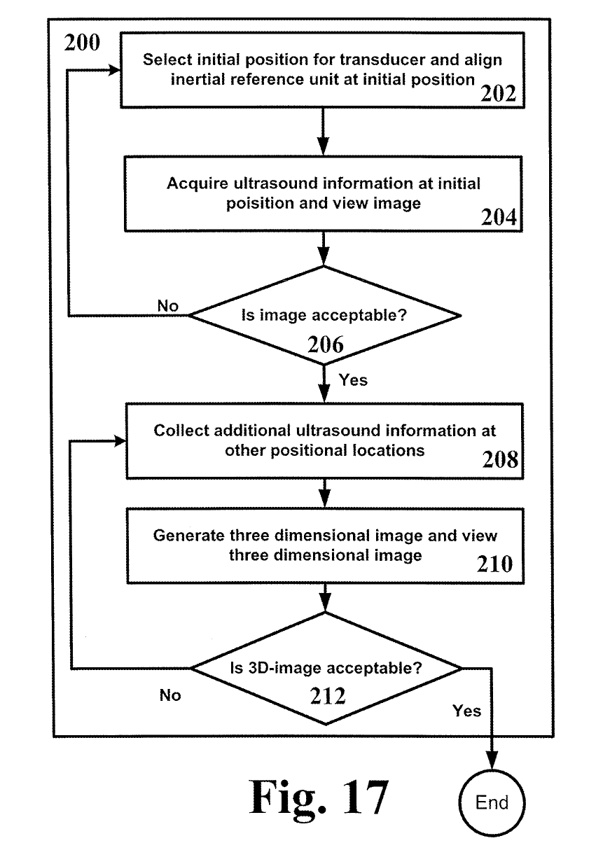

FIG. 17 is a flowchart that will be used to describe a method 200 of forming a three dimensional ultrasound image, according to an embodiment of the invention. At block 202, an initial position for an ultrasound transceiver is selected, and an inertial reference unit associated with the ultrasound transceiver is aligned at the initial position. At block 204, ultrasound image information is acquired at the initial position, and the ultrasound image is viewed. Thereafter, at decision diamond 206, an answer to the query “Is image acceptable?” is determined. Based upon a review of the image obtained at block 204, if it is determined that the initial position selected at block 202 is unsatisfactory, that is, the answer is “No”, a new initial position may be selected by cycling back to block 202. If the answer is “Yes”, that is, the initial position is satisfactory, additional ultrasound may be acquired at other positional locations, as shown at block 208. While the transceiver is moved to the other positional locations, acceleration and angular rate information is integrated along the motion path. At block 210, the ultrasound and positional information acquired at the initial and at the other additional locations is integrated to generate one or more three-dimensional images, which may be visually examined. Then, at decision block 212, if the one or more ultrasound images are determined to be unsatisfactory, then the method 200 returns to block 208, whereupon different and/or additional ultrasound information may be acquired. If the ultrasound image is determined to be acceptable, the method 200 ends, as also shown by exciting method 200 via the affirmative route from decision block 212. In alternate embodiments one or more of the foregoing method steps are omitted. In other embodiments, additional steps may be included.

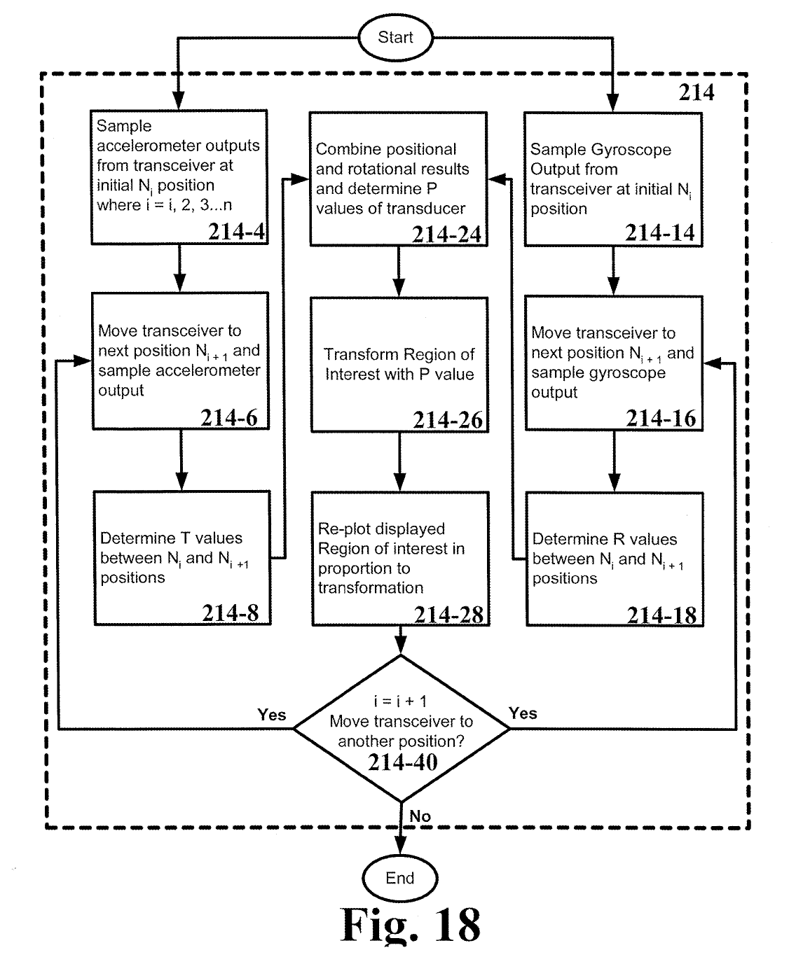

FIG. 18 is flowchart that will be used to further describe the method 200 of FIG. 17. In particular, FIG. 18 will be used to describe a method 214 of determining a position of an ultrasound transceiver, according to another embodiment of the invention. The method 200 includes blocks 214-4 and 214-14, which may be simultaneously executed, or independently executed. In general, however, it understood that motions of the transceiver relative to a patient include translations and rotations of the transceiver relative to the initial location. At block 214-4, translational signals from an accelerometer portion of the inertial reference unit are sampled at an initial position Nᵢ. At block 214-6, the transceiver is moved to another position Nᵢ₊₁, and translational signals are continuously or intermittently sampled from the accelerometer portion as the transceiver is moved from the position Nᵢ to the position Nᵢ₊₁. At block 214-8, a translational vector T is calculated for the positional location Nᵢ₊₁ by integrating the translational signals. At block 214-14, rotational rate signals obtained from the inertial reference unit are sampled at the position Nᵢ. At block 214-16, the transceiver is moved, and rotational rate signals are again continuously sampled as the transceiver is moved to the position Nᵢ₊₁. The rotational rate signals are integrated as the transceiver is moved so that rotational angles for the transceiver may be generated. Accordingly, at block 214-18, respective rotational transformation matrices Rₓ(α), R_{y}(β) and R_{z}(γ) are calculated based upon the generated rotational angles as follows:

$${R}_{x} ( α ) = [ \begin{matrix}1 & 0 & 0 \\ 0 & cos α & sin α \\ 0 & - sin α & cos α\end{matrix} ]$$

$${R}_{y} ( β ) = [ \begin{matrix}cos β & 0 & - sin β \\ 0 & 1 & 0 \\ sin β & 0 & cos β\end{matrix} ]$$

$${R}_{z} ( γ ) = [ \begin{matrix}cos γ & sin γ & 0 \\ - sin γ & cos γ & 0 \\ 0 & 0 & 1\end{matrix} ]$$

A three-dimensional rotational matrix R may then be calculated by forming a product of the rotational transformation matrices Rₓ(α), R_{y}(β) and R_{z}(γ) so that R=Rₓ(a)×R_{y}(β)×R_{z}(γ). At block 214-24, the translational vector T and the rotational matrix R obtained from block 214-8 and block 214-18, respectively, are combined so that a positional vector P may be defined for the transceiver, so that Pᵢ₊₁=RPᵢ+T. In alternate embodiments one or more of the foregoing method steps are omitted. In other embodiments, additional steps may be included.

While the preferred embodiment of the invention has been illustrated and described, as noted above, many changes can be made without departing from the spirit and scope of the invention. For example, other uses of the invention include determining the areas and volumes of the prostate, heart, bladder, and other organs and body regions of clinical interest as the images are updated by the ultrasound inertial reference system. Accordingly, the scope of the invention is not limited by the disclosure of the preferred embodiment.

We claim:

1. An ultrasound imaging system, comprising:
an ultrasound unit configured to ultrasonically scan at least one plane within a region of interest in a subject and generate imaging information from the scan;
an inertial reference unit configured to detect relative position of the ultrasound unit, during at least one scan of at least one plane; and
a processing unit configured to receive the imaging information and the detected corresponding relative positions and,
based on the received imaging information and relative position, operable to generate at least one image of the region of interest.

2. The imaging system of claim 1, wherein the inertial reference unit further comprises a first device that is configured to sense an acceleration of the ultrasound unit, and a second device that is configured to detect an angular rate of rotation of the ultrasound unit.

3. The imaging system of claim 2, wherein the first device includes an accelerometer that is responsive to at least one acceleration in a selected direction, and further wherein the second device is responsive to at least one angular rate of rotation about a selected axis.

4. The imaging system of claim 1, wherein the inertial reference unit is fixedly coupled to the ultrasound unit.

5. The imaging system of claim 1, wherein the inertial reference unit is removably coupled to the ultrasound unit.

6. The imaging system of claim 1, wherein the ultrasound unit further comprises a plurality of elements that are configured to be selectively excited to scan a plane within the region of interest.

7. The imaging system of claim 1, wherein the ultrasound unit further comprises an actuator coupled to an array of piezoelectric elements that is configured to rotate the array about a selected axis.

8. The imaging system of claim 1, wherein the processing unit is a mainframe processor that is spaced apart from the ultrasound unit.

9. The imaging system of claim 1, wherein at least the processing unit and the ultrasound are incorporated in a common unit.

10. The imaging system of claim 1, wherein the inertial reference unit further comprises a micro-electromechanical system (MEMs) based inertial reference unit.

11. A method of imaging a bodily portion in a subject, comprising:
ultrasonically scanning a first selected plane in the subject using an ultrasound scanning device to generate a first planar ultrasound data set;
determining a position of the first selected plane by accessing an inertial reference unit coupled to the ultrasound scanning device;
ultrasonically scanning a second selected plane in the subject to generate a second planar ultrasound data set;
determining a position of the second selected plane; and
processing the first data set and the position of the first selected plane and the second data set and the position of the second selected plane to generate a three-dimensional image of the bodily portion.

12. The method of claim 11, wherein determining a position further comprises determining at least one of an acceleration and an angular rate of rotation of the ultrasound scanning device.

13. The method of claim 12, wherein processing the first data set and the position of the first selected plane and the second data set and the position of the second selected plane further comprises integrating the at least one of an acceleration and an angular rate of rotation of the ultrasound scanning device.

14. The method of claim 11, wherein determining a position of the first selected plane further comprises aligning the inertial reference unit to establish a first reference position.

15. The method of claim 14, wherein aligning the inertial reference unit to establish a first reference position further comprises establishing an origin for the ultrasound scans.

16. A ultrasound system configured for hand held operation, comprising:
an ultrasound transceiver having an outwardly extending handle suitably configured to permit a user to manipulate the transceiver relative to a subject;
an inertial reference unit coupled to the transceiver that is operable to determine selected positions of the transceiver as it is moved; and
a processing unit coupled to the ultrasound transceiver and the inertial reference unit that is configured to process ultrasound data acquired from the subject and to process positional data and generate an ultrasound image therefrom.

17. The system of claim 16, wherein the inertial reference unit is removably coupled to the ultrasound transceiver.

18. The system of claim 16, wherein the processing unit is configured to receive planar ultrasound scans from the ultrasound transceiver and corresponding positional data from the inertial reference unit and generate a three dimensional ultrasound image.

19. The system of claim 16, wherein the ultrasound transceiver further comprises a suitable data interface that permits ultrasound data to be communicated to the processing unit.

20. The system of claim 19, wherein the data interface includes one of a universal serial bus (USB) and a FIREWIRE interface.

21. The system of claim 19, wherein the data interface includes a wireless data interface.

22. The system of 19, further comprising a cradle configured to receive the transceiver, further wherein the cradle includes the data interface.
